(19) United States
(12) Patent Application Publication
(10) Pub. No.: US 2015/0238144 A1
(43) Pub. Date: Aug. 27, 2015

(54) Method for Calibrating an Expandable Means of a Medical Device and Method for Monitoring the Pressure Exerted by the Interior Wall of a Biological Channel

(71) Applicant: LUNGUARD LTD., Omer (IL)

(72) Inventor: Ofer Pintel, Matan (IL)

(21) Appl. No.: 14/427,299

(22) PCT Filed: Jul. 15, 2013
(86) PCT No.: PCT/IL2013/050597

§ 371 Date: Mar. 10, 2015

Related U.S. Application Data
(60) Provisional application No. 61/699,326, filed on Sep. 11, 2012.

(51) Int. Cl. A61B 5/00

(52) U.S. Cl. CPC A61B 5/6885; A61B 5/4233; A61B 5/6853; A61B 2560/0228

(57) ABSTRACT

The present invention relates to a method for determining the working pressure (PW) of an expandable means placed in the lumen of a biological channel of diameter (d) such that the expandable means exerts a maximum pressure (P_{Max}) on the interior wall of the biological channel without causing damage to the tissues of the channel. The invention further provides a method for determining the contact pressure (PC) of an expandable means placed in the lumen of a biological channel such that the expandable means comes into full contact with the interior wall of the biological channel. The invention also provides a method for the real-time monitoring of the pressure exerted by the interior wall of a biological channel.

### FIELD OF THE INVENTION

The present invention relates to the field of medical devices. More specifically, the invention relates to methods for calibrating the expandable means of a medical device located in the lumen of a biological channel so as to avoid harming the tissues surrounding it, and methods for determining and/or monitoring the pressure exerted by the interior wall of a biological channel.

### BACKGROUND OF THE INVENTION

Modern medical devices use expandable means (EM) and in particular inflatable balloons for achieving different operations. Examples of such devices can be found for instance in WO 2010/016054.

WO 2010/016054, which is incorporated herein by reference, relates to an enteral feeding device that enables the administration of nutritive solutions directly into the stomach of a patient. The device disclosed therein significantly reduces the risks of aspirations from the alimentary tract into the respiratory system and allows deglutition of biological fluids secreted in the upper part of the digestive system into the stomach. The middle section of the feeding device of WO 2010/016054 comprises at least three expandable means surrounding a flexible tube, which can be inflated or deflated by introducing or draining a fluid into/from the internal volume of the expandable means.

Some of the purposes of the expandable means of the device disclosed in WO 2010/016054 are as follows: 1) blocking the progression of the gastrointestinal fluids in the esophagus, 2) allowing the redirection of the gastrointestinal fluids towards the stomach, and 3) enabling the swallowing of the oropharynx fluids naturally secreted by the patient.

However, the use of expandable means in the field of medical devices is not straightforward and may raise several technical issues. For instance, the inflated/deflated status of the expandable means should be carefully monitored during the introduction or removal of the medical device into the body of the patient as well as during its use within the body of the patient. Moreover, damages to the tissues belonging to the interior wall of the biological channel wherein the medical device has been introduced should be avoided. To the knowledge of the inventors, there is to date no available method for calibrating the working pressure of an expandable means when placed in the lumen of a biological channel. Furthermore, there is currently a need for a method for determining and/or monitoring the pressure exerted by the interior wall of a biological channel on the fluids flowing in the lumen.

Therefore, it is an object of the invention to provide a method for calibrating the working pressure of an expandable means of a medical device placed in the lumen of a biological channel.

It is another object of the invention to provide a method for determining and/or monitoring the pressure exerted by the interior wall of a biological channel.

Further purposes and advantages of this invention will appear as the description proceeds.

### SUMMARY OF THE INVENTION

In a first aspect, the present invention provides a method for determining the working pressure (P_{W}) of an expandable means placed in the lumen of a biological channel of diameter (d) such that said expandable means exerts a maximum pressure (P_{Max}) on the interior wall of said biological channel without causing damage to the tissues of said channel, said method comprising the steps of:
a) obtaining a first “in vitro” calibration curve (IVIT1) of said expandable means, which shows the pressure of said expandable means as a function of the volume of fluid introduced therein, when no external constraints are applied to said expandable means;
b) obtaining a second “in vitro” calibration curve (IVIT2) of said expandable means, which shows the pressure of said expandable means as a function of the volume of fluid introduced therein, when said expandable means is placed inside a rigid tube having said diameter (d);
c) obtaining an “in vivo” calibration curve (IVI) of said expandable means, which shows the pressure of said expandable means as a function the volume of fluid introduced therein, when said expandable means is placed in the lumen of a biological channel of a patient;
d) adjusting the data of the three curves obtained in steps a)-c) so as to obtain a point characterized by a pressure value P_{C} and a volume V_{C} corresponding to the pressure and volume at which the expandable means comes into full contact with the interior wall of said biological channel;
e) determining the pressure P_{S} by adding P_{Max} to P_{C};
f) determining the volume V_{S} by reporting the pressure P_{S} on the calibration curve IVIT2;
g) determining the volume V_{E} by reporting the pressure P_{S} on the calibration curve IVI;
h) determining the volume V_{Δ} by subtracting V_{S} from V_{E};
i) determining P_{Δ} by reporting V_{Δ} on the calibration curve IVIT1, with P_{C}/V_{C} as reference; and
j) determining P_{W} by adding the value of P_{C}, P_{Δ} and P_{Max}.

In a specific embodiment of the method described above, the biological channel belongs to the blood circulation system, the digestive system, the respiratory system, the urinary system or the reproductive system.

In another aspect, the invention provides a method for determining the contact pressure (P_{C}) of an expandable means placed in the lumen of a biological channel such that said expandable means comes into full contact with the interior wall of said biological channel, said method comprising the steps of:
a) obtaining an “in vitro” calibration curve (IVIT1) of said expandable means, which shows the pressure of said expandable means as a function of the volume of fluid introduced therein, when no external constraints are applied to said expandable means;
b) obtaining an “in vivo” calibration curve (IVI) of said expandable means, which shows the pressure of said expandable means as a function the volume of fluid introduced therein, when said expandable means is placed in the lumen of a biological channel of a patient; and
c) adjusting the data of the curves obtained in steps a) and b) so as to obtain a point characterized by a pressure value P_{C} and a volume V_{C} corresponding to the pressure and volume at which the expandable means comes into full contact with the interior wall of said biological channel.

In a further aspect, the invention provides a method for the real-time monitoring of the pressure exerted by the interior wall of a biological channel, said method comprising the following steps:
a) introducing at least one expandable means in the lumen of said biological channel;
b) determining the contact pressure (P_{C}) of each of said at least one expandable means following the method of claim 3;
c) inflating each of said at least one expandable means to a pressure corresponding to its contact pressure P_{C}; and
d) monitoring in real time the pressure exerted by the interior wall of said biological channel by measuring the pressure exerted on said at least one expandable means.

In a specific embodiment of the method described above, the biological channel is the esophagus.

In another specific embodiment of the method described above, the pressure exerted by the esophagus is used for determining the transpulmonary pressure.

### BRIEF DESCRIPTION OF THE DRAWINGS

The above and other characteristics and advantages of the invention will be more readily apparent through the following examples, and with reference to the appended drawings, wherein:

FIG. 1 is a graph showing three calibration curves (pressure measured as a function of volume of fluid injected) obtained for a specific expandable means, as well as the different critical points used in an embodiment of the method of the invention for calibrating this specific expandable means; the first in vitro calibration curve is represented by IVT1 (▴), the second in vitro calibration is represented by IVT2 (), and the in vivo calibration curve is represented by IVI (▪).

FIG. 2 is a scheme representing an embodiment of a method for the real-time monitoring of the transpulmonary pressure with an expandable means; PTP corresponds to the transpulmonary pressure; PAW corresponds to the tracheal air pressure; and PES corresponds to the esophageal pressure.

### DETAILED DESCRIPTION

Expandable means used with medical devices, such as inflatable balloons, are made of different material such as silicon, polyurethane, PVC and alike. While the manufacturing processes are developed to ensure that all the characteristics of the elements of a medical device are reproducible from one device to the other, it is technically very difficult to guarantee that elements such as expandable means will have exactly the same configurations and the exact physical characteristics (thickness, elasticity, self tension when inflated etc.). Furthermore, while the average value of the diameter, rigidity or flexibility of a biological channel such as the trachea, the esophagus, aorta, or any other physiological lumen in the body can be found in the art, the value may vary from patient to patient according to several factors such as age, genetic background or medical antecedents. Therefore, there is a need for a method that enables determining the working pressure of an expandable means when placed in the lumen of a biological channel, so it fulfills its role in said biological channel without damaging the surrounding tissues.

In a first aspect, the present invention aims to provide a method for calibrating an expandable means placed inside the biological channel of a patient and determining its working pressure (P_{W}). The expandable means is preferably placed within the lumen of a biological channel that belongs to the blood circulation system (heart, artery, vein), the digestive system (esophagus, stomach, duodenum, small intestine, large intestine, anus), the respiratory system (trachea, bronchi), the urinary system (kidney, ureter, bladder, urethra) or the reproductive system (vas deferens, ejaculatory duct, vagina, uterus, fallopian tube). The methods of the present invention may be also applied to biological channels belonging to animal organisms other than human, plant organisms, or other tubular structure from the industry.

The maximum pressure that can be applied for a long period of time on epithelial tissues without damaging them is known in the art. In most cases, this pressure should serve as a target pressure or maximum pressure (P_{Max}) that can be applied continuously by an expandable means on the interior wall of the channel. However, the pressure of the fluid measured within the expandable means by external pressure sensors does not correspond to the pressure effectively applied by the expandable means on the wall of the biological channel. Indeed, it was found by the inventors that several additional factors should be considered when calibrating the expandable means, such as the pressure developed to inflate the expandable means up to the full contact with the wall of the biological channel (called hereafter the “contact pressure” P_{c}), and additional factors such as the pressure applied to compensate the deformation of the biological channel (P_{Δ}). The contact pressure P_{C} depends on severable variables inherent to the expandable means such as the material, thickness, elasticity, diameter, or shape. The pressure P_{Δ} merely reflects the pressure which causes the flexible biological channel to enlarge its diameter, but is not a pressure directly applied onto the wall of the channel.

In summary, the pressure relative to the expandable means as measured by the external sensors (referred herein as the working pressure (P_{W})) can be defined as follows:

P_{W}=P_{c}+P_{Max}+P_{Δ}

wherein
P_{W} corresponds to the working pressure as measured by the external sensors;
Pc corresponds to the contact pressure, in other words the pressure used to inflate the expandable means and bring it into the full contact with the wall of the channel (no effect on the tissues);
P_{Max} corresponds to the maximum pressure that can be continuously applied on the wall of the biological channel; and
P_{Δ} corresponds to the pressure due to additional factors, such as the deformation of the biological channel.

The working pressure (P_{W}) should be calibrated for each expandable means independently, so that the pressure continuously applied to the wall of the biological channel by said expandable means does not exceed the maximum pressure (P_{Max}) described in the literature, above which damages are made to the tissues.

The inventors surprisingly found that the calibration of P_{W} can be achieved by using the following method. It should be emphasized that all the tests are performed on a medical device comprising the expandable means to be calibrated. For the sake of conciseness, the term “expandable means” as used thereafter can be understood as either an expandable means alone or an expandable means located on a medical device.

During the manufacturing process of the medical device, two “in vitro” tests are made on the expandable means. The first in vitro test comprises the following steps:

1) the expandable means is deflated and a volume of fluid is gradually injected in the expandable means until a predetermined volume threshold is reached;
2) the pressure inside the expandable means is recorded as a function of the volume of fluid injected and a calibration curve (IVT1) is recorded; and
3) the recorded data are transferred on a digital media (e.g. electronic chip) associated with the expandable means.

The second in vitro test comprises the following steps:

1) the expandable means is placed in a rigid tubular structure having a diameter corresponding to the target biological channel (e.g. average diameter of an esophagus);
2) the expandable means is deflated and a volume of fluid is gradually injected in the expandable means until a predetermined volume threshold is reached;
3) the pressure inside the expandable means is recorded as a function of the volume of fluid injected and a calibration curve (IVT2) is recorded; and
4) the recorded data are transferred on a digital media (e.g. electronic chip) associated with the expandable means.

The data recorded are used for determining the working pressure of the expandable means after the in vivo test has been performed.

The in vivo test comprises the following steps:

1) the expandable means is introduced in the lumen of the biological channel of the patient;
2) the expandable means is deflated and a volume of fluid is gradually injected in the expandable means until a predetermined volume threshold is reached; and
3) the pressure inside the expandable means is recorded as a function of the volume of fluid injected and a calibration curve (IVI) is recorded.

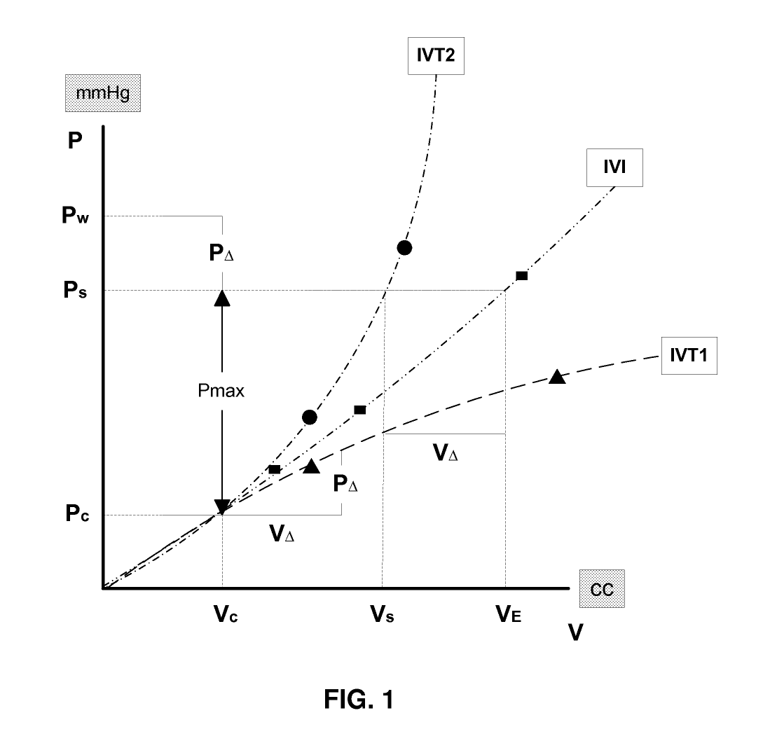

The data obtained from the in vivo test and those obtained from the two in vitro tests are then automatically treated and analyzed via a software program. The data of the three curves are adjusted and the best fit between the three curves is determined (see FIG. 1). By doing so, a characteristic point appears, above which a discrepancy between the values of the pressures obtained in the three different curves is observed. This characteristic point corresponds to the pressure P_{C} and the volume V_{C} at which the expandable means comes into full contact with the interior wall of the biological channel/rigid tube. Up to the pressure P_{C}, the pressure measured in the expandable means represents the pressure needed to inflate the expandable means and bring it into full contact with the surrounding wall/tube with no actual pressure effect on the tissue. To the pressure P_{C}, the pressure P_{Max} is added in order to reach a pressure P_{S}. Reporting the value P_{S} to the calibration curve IVT2 () gives a volume V_{S}, which is the volume necessary to apply P_{Max} to the wall of the rigid tube in the second in vitro experiment (as described above). Similarly, reporting the value P_{S} to the calibration curve IVI (▪) gives the volume V_{E}. The difference between the volume V_{E} and the volume V_{S} corresponds to a volume V_{Δ}, which reflects the volume of fluid injected in the expandable means to enlarge the flexible biological channel surrounding. In order to determine the pressure P_{Δ} which is the pressure used to inflate the expandable means in the biological channel until it applies a pressure on the wall, the volume V_{Δ} is reported to the calibration curve IVT1 (▴), taking P_{C}/V_{C} as a reference (see FIG. 1).

The value of the working pressure P_{W} is therefore determined as follows:

P_{W}=P_{c}+P_{Max}+P_{Δ}

wherein
P_{W} corresponds to the working pressure as measured by the external sensors;
Pc corresponds to the contact pressure, in other words the pressure used to inflate the expandable means and bring it into the full contact with the wall of the channel (no effect on the tissues);
P_{Max} corresponds to the maximum pressure that can be continuously applied on the wall of the biological channel; and
P_{Δ} corresponds to the pressure due to additional factors, such as the deformation of the biological channel.

As it can be understood from the above, the working pressure P_{W} is characteristic of a specific expandable means at a certain position within the body of the patient, and corresponds to the optimal working pressure of the expandable means guarantying its full functionality without damaging the surrounding tissues.

A further aspect of the invention provides a method for monitoring the pressure exerted by the interior wall of a biological channel. By applying the calibration method described above, the working pressure at which the expandable means is in contact with the wall of the biological channel (P_{C}) can be determined. When the expandable means is inflated at a pressure value of P_{C}, any deformation of the biological channel that would have resulted in the application of a pressure on the material flowing inside the biological channel is applied instead on the inflated expandable means. The pressure exerted on the expandable means can be reported to a central unit and may help monitoring the pressure exerted by the interior wall of a biological channel in real time.

This is for instance of particular relevance when monitoring the transpulmonary pressure of a treated patient (see Example 2). The transpulmonary pressure s calculated as follows:

P_{TP}=P_{AW}−P_{ES}

wherein
P_{TP} corresponds to the transpulmonary pressure;
P_{AW} corresponds to the tracheal air pressure; and
P_{ES} corresponds to the esophageal pressure.

The following examples, which further describe the invention, are offered by way of illustration and are not intended to limit the invention in any manner.

### Example 1

### Method for Determining the Optimal Working Pressure of an Expandable Means in a Human Esophagus

The present example reports the calibration of an expandable means belonging to a device similar to the one described in WO 2010/016054. The pressure inside the expandable means is measured via a control and monitoring unit (CMU) as described in WO 2010/01604. The presently described method aims to determine the optimal working pressure (P_{W}) of the expandable means when located in the esophagus of a treated patient. As discussed above, determining P_{W} is important to insure that the expandable means achieve the desired function without damaging the tissues of the esophagus during the treatment. In the present example, a maximum pressure P_{Max} of 30 mmHg is to be applied by the expandable means on the internal wall of the esophagus.

Phase 1: In vitro calibration test 1 (IVT1)
1) the expendable means is deflated;
2) the expendable means is gradually inflated by injecting a volume of 0.2 cc of air until a total volume of 5 cc is reached;
3) when a total volume of 5 cc has been injected in the expendable means, the calibration curve reporting the pressure as a function of the volume of air is generated; and
4) the graph obtained in step 3 is recorded on an electronic chip which also contains information relative to the identification number of the expandable means.

This calibration curve corresponds to the “in vitro” test number 1 (IVT1).

### Phase 2: In Vitro Calibration Test 2 (IVT2)

1) the expandable means is placed in a rigid tubular structure having a diameter corresponding to the average diameter of an esophagus, namely 14 mm;
2) the expendable means is deflated;
3) the expendable means is gradually inflated by injecting a volume of 0.2 cc of air until a total volume of 5 cc is reached;
4) when a total volume of 5 cc has been injected in the expendable means, the calibration curve reporting the pressure as a function of the volume of air is generated; and
5) the graph obtained in step 4 is recorded on an electronic chip which also contains information relative to the identification number of the expandable means.

This graph corresponds to the “in vitro” test number 2 (IVT2).

### Phase 3: In Vivo Calibration (IVI)

1) the expandable means is placed in the patient's esophagus;
2) the expendable means is deflated;
3) the expendable means is gradually inflated by injecting a volume of 0.2 cc of air until a total volume of 5 cc is reached;
4) when a total volume of 5 cc has been injected in the expendable means, the calibration curve reporting the pressure as a function of the volume of air is generated; and
5) the graph obtained in step 4 is recorded and stored in the CMU.

### Phase 4: Adjusting the Calibration Curves

1) the expandable means is identified via its identification number by the CMU;
2) the CMU is importing the data corresponding to the two in vitro tests performed in Phase 1 and Phase 2 described above;
3) a software present in the CMU is adjusting the 3 graphs obtained during Phases 1-3 (e.g. by calculating and comparing the derivative of each point of the graphs for a same specific volume);
4) once adjusted, the system determines the pressure corresponding to the pressure at the contact point of the expandable means on the internal esophagus wall/rigid tube wall (P_{C}).

### Phase 5: Determining the Optimal Working Pressure

1) a pressure value P_{S} is determined by adding the pressure P_{Max} (30 mmHg) to the pressure P_{C} (in this case 10 mmHg).
2) the pressure P_{S} is reported on the calibration curve obtained from the data of the second in vitro experiment to find V_{S};
3) the pressure P_{S} is reported on the calibration curve obtained from the data of the in vivo experiment to find V_{E};
4) the volume V_{Δ} is calculating by the difference V_{E}−V_{S};
5) the value of V_{Δ} is reported on the calibration curve obtained from the data of the first in vitro experiment and the pressure P_{Δ} is determined; and
6) eventually, the optimal P_{W} is determined by effecting the following operation:

P_{W}=P_{C}+P_{MAX}+P_{Δ}

In a particular case (data not shown), the pressure at the contact point (P_{C}) has been determined at 11 mmHg, P_{Max} at 30 mmHg and P_{Δ} at 11 mmHg. Therefore, the optimal working pressure P_{W} in this case has been set to 52 mmHg. In conclusion, when this specific expandable means is inserted inside the esophagus, the working pressure should be about 52 mmHg so that said expandable means effectively applies a continuous pressure on the interior wall of the esophagus of about 30 mmHg, effectively closing the lumen space without damaging the surrounding tissues.

### Example 2

### Method for the Real-Time Monitoring of the Transpulmonary Pressure

Ventilated patients in the intensive care unite are often needed to be monitored for their esophageal pressure during ventilator setting especially during peep adjustment. Pleural pressure can be measured via an esophageal balloon catheter and allows for calculation of the transpulmonary pressure which is the difference between the alveolar pressure (the pressure measured at the airway opening when flow is stopped) and pleural pressure. The measurements are done in the upright position and the expandable means is placed in lower third of the esophagus. Cardiac oscillations are ignored. Because the pressure of an expandable can be measured, the esophageal pressure applied on the expandable can be determined by using the expandable means as a manometer.

In the present example, a device similar to the one in WO 2010/016054 is employed in the esophagus of a patient. All three balloons are deflated and then inflated simultaneously to reach their respective contact pressures P_{C}(1), P_{C}(2) and P_{C}(3). As it can be understood from the description, the values P_{C}(1), P_{C}(2) and P_{C}(3), while similar in some cases, are usually distinct since each one of them corresponds to a specific expandable means (having inherent properties) at a specific position of the esophagus. The transpulmonary pressure s calculated as follows:

P_{TP}=P_{AW}−P_{ES}

wherein
P_{TP} corresponds to the transpulmonary pressure;
P_{AW} corresponds to the tracheal air pressure; and
P_{ES} corresponds to the esophageal pressure with P_{ES}=P_{M}−P_{C}, wherein
P_{M} corresponds to the pressure measured within the expendable means; and
P_{C} corresponds to the contact pressure of the same expandable means

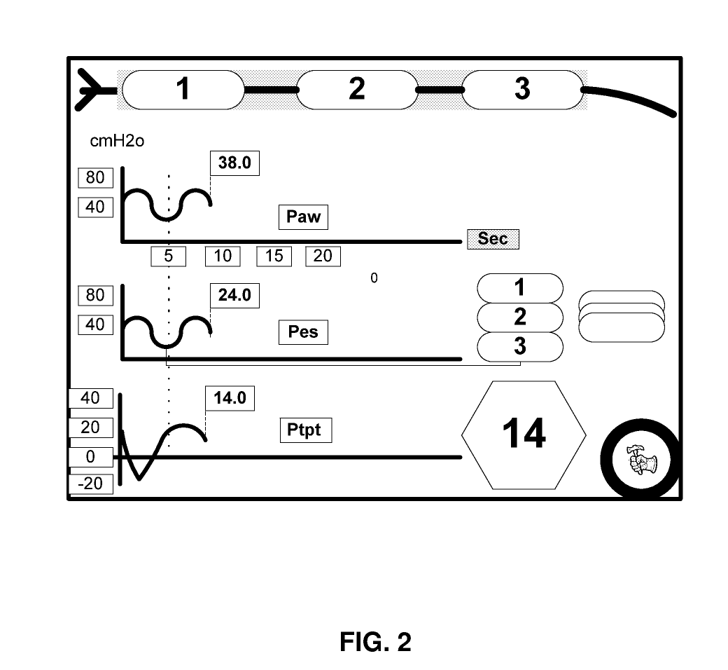

As shown in FIG. 2, the tracheal air pressure (P_{AW}) is obtained from the data transferred by the tracheal tube. The esophageal pressure (P_{ES}) is determined in real-time by measuring at least one of the pressure values of expandable means 1-3, which have been inflated at their respective contact pressure and are used to measure the pressure variations in the lumen of the esophagus. The average of the values reported by the expandable means (or any other combinations) is also considered as a possible embodiment. In the present example (FIG. 3), P_{AW} as measured is 38 cmH₂O and P_{ES} is the average value reported by the three expandable means and corresponds to 24 cmH2O. Therefore, the transpulmonary pressure P_{TP} corresponds in this case to 14 cmH₂O (or 19 mmHg, when considering that 1 cmH₂O=1.359 mmHg).

It is clear from the above that the present method allows real-time monitoring of the transpulmonary pressure via the use of at least one expandable means placed in the esophagus and further data obtained from a tracheal tube.

Although embodiments of the invention have been described by way of illustration, it will be understood that the invention may be carried out with many variations, modifications, and adaptations, without exceeding the scope of the claims.

1. A method for determining the working pressure (P_{W}) of an expandable means placed in the lumen of a biological channel of diameter (d) such that said expandable means exerts a maximum pressure (P_{Max}) on the interior wall of said biological channel without causing damage to the tissues of said channel, said method comprising the steps of:
a) obtaining a first “in vitro” calibration curve (IVIT1) of said expandable means, which shows the pressure of said expandable means as a function of the volume of fluid introduced therein, when no external constraints are applied to said expandable means;
b) obtaining a second “in vitro” calibration curve (IVIT2) of said expandable means, which shows the pressure of said expandable means as a function of the volume of fluid introduced therein, when said expandable means is placed inside a rigid tube having said diameter (d);
c) obtaining an “in vivo” calibration curve (IVI) of said expandable means, which shows the pressure of said expandable means as a function the volume of fluid introduced therein, when said expandable means is placed in the lumen of a biological channel of a patient;
d) adjusting the data of the three curves obtained in steps a)-c) so as to obtain a point characterized by a pressure value P_{C} and a volume V_{C} corresponding to the pressure and volume at which the expandable means comes into full contact with the interior wall of said biological channel;
e) determining the pressure P_{S} by adding P_{Max} to P_{C};
f) determining the volume V_{S} by reporting the pressure P_{S} on the calibration curve IVIT2;
g) determining the volume V_{E} by reporting the pressure P_{S} on the calibration curve IVI;
h) determining the volume V_{Δ} by subtracting V_{S} from V_{E};
i) determining P_{Δ} by reporting V_{Δ} on the calibration curve IVIT1, with P_{C}/V_{C} as reference; and
j) determining P_{W} by adding the value of P_{C}, P_{Δ} and P_{Max}.

2. The method according to claim 1, wherein said biological channel belongs to the blood circulation system, the digestive system, the respiratory system, the urinary system or the reproductive system.

3. A method for determining the contact pressure (P_{C}) of an expandable means placed in the lumen of a biological channel such that said expandable means comes into full contact with the interior wall of said biological channel, said method comprising the steps of:
a) obtaining an “in vitro” calibration curve (IVIT1) of said expandable means, which shows the pressure of said expandable means as a function of the volume of fluid introduced therein, when no external constraints are applied to said expandable means;
b) obtaining an “in vivo” calibration curve (IVI) of said expandable means, which shows the pressure of said expandable means as a function the volume of fluid introduced therein, when said expandable means is placed in the lumen of a biological channel of a patient; and
c) adjusting the data of the curves obtained in steps a) and b) so as to obtain a point characterized by a pressure value P_{C} and a volume V_{C} corresponding to the pressure and volume at which the expandable means comes into full contact with the interior wall of said biological channel.

4. A method for the real-time monitoring of the pressure exerted by the interior wall of a biological channel, said method comprising the following steps:
a) introducing at least one expandable means in the lumen of said biological channel;
b) determining the contact pressure (P_{C}) of each of said at least one expandable means following the method of claim 3;
c) inflating each of said at least one expandable means to a pressure corresponding to its contact pressure P_{C}; and
d) monitoring in real time the pressure exerted by the interior wall of said biological channel by measuring the pressure exerted on said at least one expandable means.

5. A method according to claim 4, wherein said biological channel is the esophagus.

6. A method according to claim 5, wherein the pressure exerted by the esophagus is used for determining the transpulmonary pressure.
